(19) United States
(12) Patent Application Publication
(10) Pub. No.: US 2015/0337339 A1
(43) Pub. Date: Nov. 26, 2015

(54) Production Method of Enzyme Containing Antrodia Cinnamomea Component

(71) Applicant: HU LU DAN BIOTECH CO., LTD., Tianwei Township (TW)

(72) Inventors: Tseng Tsai CHEN, Tianwei Township (TW); Yu Chuan SU, Tianwei Township (TW); Han Shu WU, Tianwei Township (TW); Kun Yi CHEN, Tianwei Township (TW); Wen-Che LIAO, Tianwei Township (TW)

(73) Assignee: HU LU DAN BIOTECH CO., LTD., Tianwei Township (TW)

(21) Appl. No.: 14/283,001

(22) Filed: May 20, 2014

(51) Int. Cl. C12P 7/06

(52) U.S. Cl. CPC C12P 7/06

(57) ABSTRACT

A production method of enzyme containing Antrodia Cinnamomea component includes steps of: filling fruits of the season and Antrodia Cinnamomea in eighty percent full in the internal space of a container to be sealed off and adding a yeast for a first yeast fermentation for a period of 30 days; then stuffing the same container with the fruits of the season for a second yeast fermentation for a period of 30 days; injecting air into the container when alcohol level goes lower than 12% and adding an acetic acid bacteria for an acetic acid fermentation for a period of 120˜180 days. Through two times of alcoholic extraction, followed with the acetic acid extraction, the particular bioactive components of Antrodia Cinnamomea may be fully obtained and carried with the natural enzyme drinks.

### BACKGROUND

1. Field of the Invention

The present invention relates to a production method of enzyme, and in particular to a method in that Antrodia Cinnamomea is used for bioactive components extraction of triterpenoids, et cetera.

2. Related Art

Enzymes, in brief, act as a catalyst to carry out biological and chemical reactions in living organisms and mainly exist in living cells of animals and plants. Synthesized substances of cells, including proteins, carbohydrates, fats, carbon-based materials and vitamins, etc. are broken down by enzymes for living creatures to digest and maintain their lives.

As far as our human bodies are concerned, the metabolic processes of 60 trillion cells in the human body rely on enzymes to drive various chemical reactions and the substance to be catalyzed is called “substrate”. Every enzyme has its specific function, that is, only when the right enzyme finds the right substrate can biochemical reactions occur for a particular product. Enzymes must be syntheized in living cells and thus microorganisms are commonly used in production of enzymes. The currently known enzymes are over two thousands of types, functioning in complexity.

Take human bodies as an example in explaining the importance of enzymes. Human bodies take food for nutrients to supply energy to body cells, but the incoming food are of large molecules and may not be absorbed right away and in consequence, enzymes are required in breaking down nutrients for digestions so that the nutrients may function in the bodies. Howerver, health is declining with ages; the abilities to produce enzymes will go down as well, and thus regulating diet and taking supplementary food containing enzymes in high quality will become necessary.

Further, Antrodia Cinnamomea, alias “Camphor mushroom”, “the Camphor Cave mushroom”, “Antrodia mushroom”or “Red camphor” in Taiwanese folk, is endemic fungi in Taiwan, which is perennial genus fungi, belonging to Aphyllophorales, Polyporaceae. The fruiting bodies of Antrodia Cinnamomea are perennial, sessile, corky to woody, having a strong camphor aroma and varied forms in plate, bell-shaped, or horseshoe-shaped or tower. Newborn is shown as flat and bright red, and then the edges will turn to form brackets in radial and expand all around to grow. Meanwhile, the color will change to reddish brown or light brown, and there are many pores, being the richest parts in medicinal values of Antrodia Cinnamomea.

Antrodia Cinnamomea contains a lot of bioactive components, including: triterpenoids, polysaccharides (such as β-D-glucan), adenosine, vitamins (like vitamin B, niacin), proteins (including immunoglobulins), superoxide dismutase (SOD), trace elements (eg: calcium, phosphorus, germanium), nucleic acids, steroids and blood pressure stabilizing substances (eg antodia acid), etc. Such bioactive ingredients are considered to be effective in anti-tumor treatment, improving immunity, helping the body fight off infection,bacteria, reducing the severity of allergies, lowering high blood pressure and blood sugar, inhibiting blood platelet aggregation,keeping cholesterol levels from rising and protecting the liver.

Apart from the uses in preventing the bodies from tumors and treatment thereof, proliferation of cancer cells and transfer, Antrodia Cinnamomea may be in help eliminating malignant ascites and significantly alleviate megalgia, inappetence and side effects of Radiotherapy and Chemotherapy, etc. Many cancer patients in survey said that their life quality is magnificently improved and back to normal gradually after consuming Antrodia Cinnamomea.

β-D-glucan contained in Antrodia Cinnamomea is anti-cancer and the high amount of triterpenoids may enhance the anti-tumor effect. Medical experiments have proved that Antrodia Cinnamomea may inhibit the growth of cancerous cells of the liver. Apart from the uses in preventing the bodies from tumors and treatment thereof, proliferation of cancer cells and transfer, Antrodia Cinnamomea may be in help eliminating malignant ascites and significantly alleviate megalgia, inappetence and side effects of Radiotherapy and Chemotherapy, etc.

Cows graze to grow over one hundred kilos but for humans, grass diet will lead to malnutrition. It's because that no digestive ferment (a type of enzymes) in human bodies to break the cell walls of plants. Direct pulverization without walls breaking make extraction of nutrients inside the cell walls a big problem. And after consuming the pulverized substances, what nutrients and the amounts thereof may be absorbed by human bodies are unknown. However, due to that only a few nutrients of Antrodia Cinnamomea may be solvent in water and the most precious nutrition facts of Antrodia Cinnamomea are the integrity and diversities of triterpenoids, alcoholic extraction will abstract the sole nutrients rather than water extraction.

Further, for food themselves, food in liquid will be easier on digestion and absorption than food in solid. The tradition way in producing food enzymes is to disposit fresh fruits, vegetables and wild grass into a clean container, then pour into yeast and sugar and then stir the mixture. When yeast encounters the substrate “sugar”, hydrolysis will take place to break apart sugar to obtain energy for cell division and proliferation. When the cell division comes to saturation, yeast will automatically slow down cell dividing and proliferating and turn to convert sugars into etyhl alcohol and carbon dioxide to make wine. Therefore the enzyme producers, before cell division of the fungus body reaches saturation, will add high amounts of sugar to keep a higher level of the sugar substrate to deactivate the yeast and inhibit wine making process.

In consequence, through the traditional method to make natural enzyme drinks will not be able to carry the partiular nutrients of Antrodia Cinnamomea, such as triterpenoids with the drinks. Especially to the chemotherapy patients, who are incapable of eating in normal, the tradtionally made enzyme drinks may not supplement the diverse bioactive components of Antrodia Cinnamomea to improve immunity.

### SUMMARY

Accordingly, to solve the aforementioned defects, the objective of the present invention is to provide a production method of enzyme containing Antrodia Cinnamomea component in that alcoholic extraction by two times of yeast fermentation is used to make the particular bioactive components, like triterpenoids, to be carried with neutral enzyme drinks.

To achieve the purpose, the present invention discloses a production method of enzyme containing Antrodia Cinnamomea component. The production method includes the following steps: Step (a): Filling a sterilized and diced fruits of the season and a Antrodia Cinnamomea in eighty percent full in the internal space of a container to be sealed off and adding a yeast for a first yeast fermentation for a period of 30 days; Step (b): Then stuffing the same container with the sterilized and diced fruits of the season for a second yeast fermentation for a period of 30 days; Step (c): detecting the Ethanol concentration of the fermented liquid in the same container and, when the detected level goes lower than 12%, controlling the Step (b) by injecting air into the container and adding an acetic acid bacteria for an acetic acid fermentation for a period of 120˜180 days; Step (d): detecting the pH value of the fermented liquid in the same container; carrying out a solid-liquid separation when the value is between 3.0˜3.5; or when the value goes ≧3.5, which is not the right condition for separation, just observing and continuing the acetic acid fermentation until the value reaches the pH value between 3.0˜3.5 for the solid-liquid separation. Step (e): standing the liquid of Step (d) until maturation for a period of 30 days; removing the sediments to obtain the enzyme fluids with components of the Antrodia Cinnamomea.

Wherein, the Antrodia Cinnamomea and the fruits in Step (a) are at a weight ratio of 1:2˜028.

Wherein, a Cordyceps militaris may be further added in Step (a) with a weight ratio of 1:1˜2 between the Antrodia Cinnamomea and the Cordyceps militaris.

Wherein, a Curcuma longa may be further joined in Step (a) with a weight ratio of 1:0.6˜0.8 between the Antrodia Cinnamomea and the Curcuma longa.

Wherein, a Cordyceps militaris and a Curcuma longa may be further added in Step (a) with a weight ratio of 1:1˜20.6˜0.8 between the Antrodia Cinnamomea, the Cordyceps militaris and the Curcuma longa.

The advantage of the present invention lies in that, through the refilling of fruits for a second yeast fermentation, the effects of alcoholic extraction of Antrodia Cinnamomea may be enhanced, and accompanied with the addition of acetic acid bacteria for acetic acid extraction, the particular bioactive components of Antrodia Cinnamomea, like triterpenoids, may be fully obtained and carried with the natural enzyme drinks. Enzymes and the particular bioactive efficacy of Antrodia Cinnamomea are synergized to provide in fluids for absorption and improve the immunity of the consumers.

### BRIEF DESCRIPTION OF THE DRAWINGS

FIG. 1 is a schematic flow diagram of embodiment 1.

FIG. 2 is a schematic flow diagram of embodiment 2.

FIG. 3 is a schematic flow diagram of embodiment 3.

FIG. 4 is a schematic flow diagram of embodiment 4.

### DETAILED DESCRIPTION

Reference will now be made in detail to the preferred embodiment of the present disclosure, examples of which are illustrated for the purpose of describing the present invention and not to limit the implementation of the present invention.

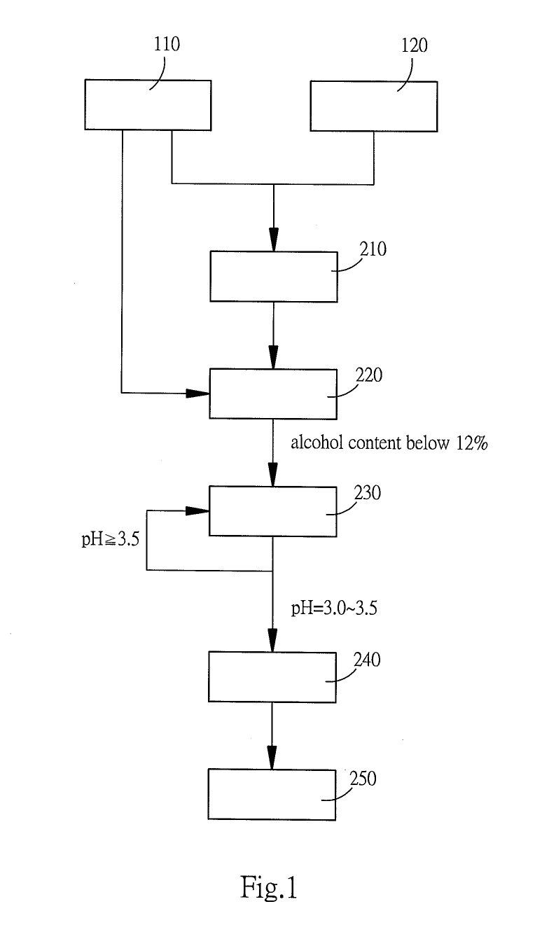

Please refer to FIG. 1, a schematic flow diagram of embodiment 1. The present invention discloses a production method of enzyme containing Antrodia Cinnamomea component, comprising steps of:

Step (a): Filling a sterilized and diced fruits 110 of the season and a Antrodia Cinnamomea 120 in eighty percent full in the internal space of a container to be sealed off, wherein the Antrodia Cinnamomea 120 and the fruits 110 are at a weight ratio of 1:20˜28, and adding a yeast for a first yeast fermentation 210 for a period of 30 days. Through an occurrence of hydrolysis between the yeast and sugar of the fruits 110, the sugar is broken apart and energy is obtained for cell division and proliferation and when the cell division comes to saturation, the yeast will automatically slow down cell dividing and proliferating and turn to convert the sugar into etyhl alcohol and carbon dioxide for a first time of alcoholic extraction of Antrodia Cinnamomea 120 using etyhl alcohol, wherein the fruits 110 may be fruits of the season, such as apples, kiwis, grapes, pineapples, dragon fruits, green papayas, roman tomatoes, guavas, pears and lemons and so on. After the fruits being cleaned and air dried, ultraviolet sterilization may be used to keep hygiene of the fruits 110 and the yeast may be any of the species suitable to be used for fruit enzymes, like Leuconostoc Mesenteroide, Lactobacillus brevis, Pediococcuscerevisiae, Lactobacillus plantarum, Lactobacillus paracasei and BRAP-01 and so forth. Attention shall be paid on that alcoholic fermentation need not oxygen, the container is required to be fully sealed to keep high level of etyhl alcohol and meanwhile to avoid contamination.

Step (b): After the first yeast fermentation 210 of the fruits 110 and the Antrodia Cinnamomea 120 in the Step (a), stuffing the same container to be sealed with the sterilized and diced fruits 110 of the season for a second yeast fermentation 220 in the sealed container for a period of 30 days. Through the refilling of the fruits 110, the level of the sugar is increased to expect more of the etyhl alcohol for the second alcoholic extraction, and the effects of alcoholic extraction of the Antrodia Cinnamomea 120 may be enhanced. Antrodia Cinnamomea contains a lot of bioactive components, including: triterpenoids, polysaccharides (such as β-D-glucan), adenosine, vitamins (such as vitamin B, niacin), proteins (including immunoglobulins), superoxide dismutase (SOD), trace elements (eg: calcium, phosphorus, germanium), nucleic acids and steroids, among which the most precious nutrition facts of Antrodia Cinnamomea are the integrity and diversities of triterpenoids. Therefore to enhance the effects of alcoholic extraction of the Antrodia Cinnamomea 120 is meant for the extraction of the integrity and diversities of triterpenoids and more of them may be carried with the enzyme.

Step (c): detecting the Ethanol concentration of the fermented liquid in the same container and, when the detected level goes lower than 12%, controlling the Step (b) of the second yeast fermentation 220 by injecting air into the container and adding an acetic acid bacteria for an acetic acid fermentation 230 for a period of 120˜180 days. The acetic acid bacteria will stop the activity of the yeast and the Ethanol concentration of the fermented liquid is controlled lower than 12%, under which the Antrodia Cinnamomea 120 undergoes the alcoholic extraction in the acetic acid fermentation 230 to further enhance the extraction effects of the Antrodia Cinnamomea 120.

Step (d): detecting the pH value of the fermented liquid in the same container and carrying out a solid-liquid separation 240 when the value is between 3.0˜3.5; or when the value goes ≧3.5, which is not the right condition for separation, just observing and continuing the acetic acid fermentation 230 until the value reaches the pH value between 3.0˜3.5 for the solid-liquid separation 240.

Step (e): leavng the liquid from the solid-liquid separation 240 in Step (d) in a process of standing to mature 250 for a period of 30 days; then removing the sediments to obtain the enzyme fluids with components of Antrodia Cinnamomea.

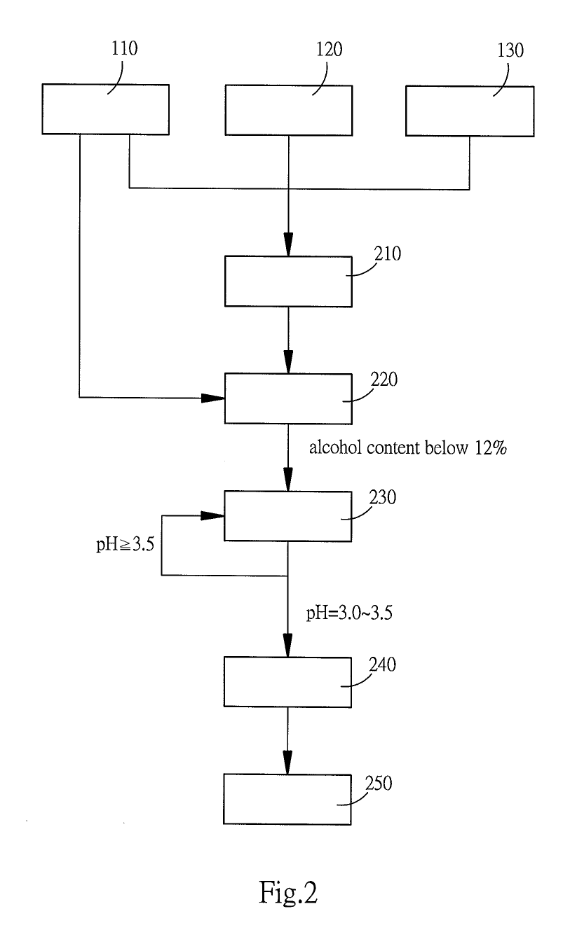

Referring to FIG. 2, in implementation, a Cordyceps militaris 130 may be further added in the Step (a) with a weight ratio of 1:1˜2 between the Antrodia Cinnamomea 120 and the Cordyceps militaris 130. The Cordyceps militaris, after identification and characterization, like Ophiocordyceps sinensis, are in the family of fungi; the efficacy and the pharmacological action of the Cordyceps militaris is also similar to the Ophiocordyceps sinensis. However, because of different parasites, the Cordyceps militaris contains sixteen kinds of amino acid and more of Cordycepin, and further with a higher existence of selenium, which is well-known as a fundamental element for life and the effective tool in warding off cancers, and of antioxidant Superoxide dismutase (SOD) than the Ophiocordyceps sinensis.

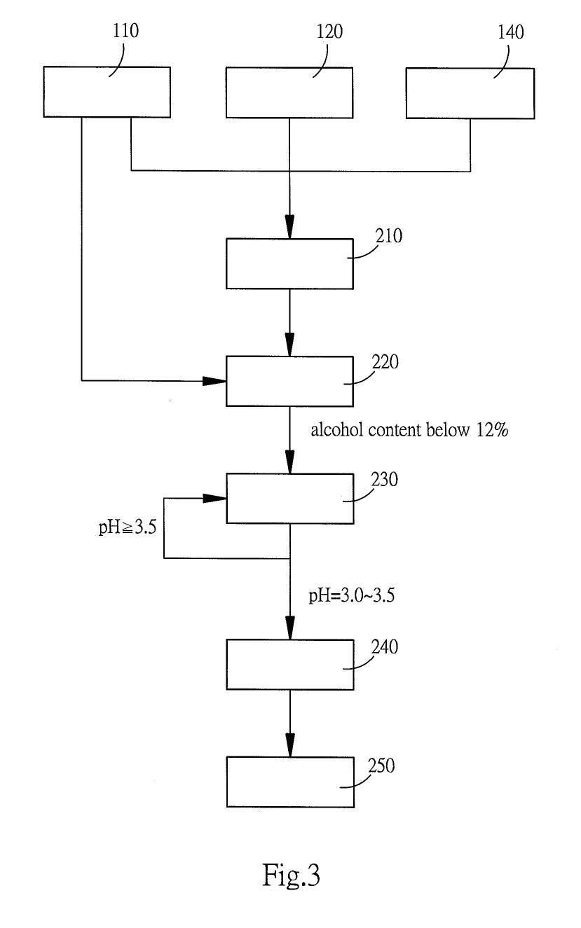

Referring to FIG. 3, in implementation, a Curcuma longa 140 may be further added in the Step (a) with a weight ratio of 1:0.6˜0.8 between the Antrodia Cinnamomea 120 and the Curcuma longa 140. The main constituent, curcumin, of the Curcuma longa has some medical benefits, as shown in the preliminary clinical trials data, of improving immunity, anti-oxidant, preventing from cancers. Through the combination of the Curcuma longa 140 and the Antrodia Cinnamomea 120, the effects of enzyme on improving immunity may be enhanced.

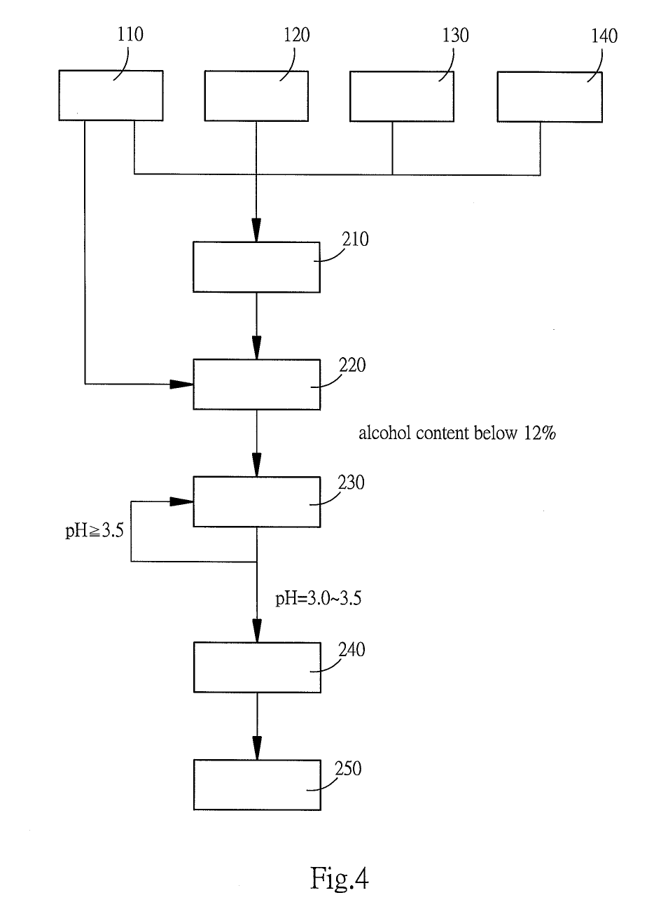

Referring to FIG. 4, in implementation, a Cordyceps militaris 130′ and a Curcuma longa 140′ may be further added in the Step (a) with a weight ratio of 1:1˜2:0.6˜0.8 among the Antrodia Cinnamomea 120, the Cordyceps militaris 130′ and the Curcuma longa 140′. Through the way that the Curcuma longa 140′ and the Cordyceps militaris 130′ join with the Antrodia Cinnamomea 120, the effect of enzyme on immunity may be strengthened.

The previous description of the preferred embodiments is provided to further describe the present invention, not intended to limit the present invention. Any modification of the disclosure will be construed as being included in the present invention.

What is claimed is:

1. A production method of enzyme containing Antrodia Cinnamomea component, comprising steps of:
Step (a): filling a sterilized and diced fruits of the season and a Antrodia Cinnamomea in eighty percent full in the internal space of a container to be sealed off and adding a yeast for a first yeast fermentation for a period of 30 days;
Step (b): then stuffing the same container with the sterilized and diced fruits of the season for a second yeast fermentation for a period of 30 days;
Step (c): detecting the Ethanol concentration of the fermented liquid in the same container and, when the detected level goes lower than 12%, controlling the Step (b) by injecting air into the container and adding an acetic acid bacteria for an acetic acid fermentation for a period of 120˜180 days;
Step (d): detecting the pH value of the fermented liquid in the same container; carrying out a solid-liquid separation when the value is between 3.0˜3.5;
Step (e): standing the liquid of the Step (d) until maturation for a period of 30 days; removing the sediments thereof to obtain the enzyme fluids with components of the Antrodia Cinnamomea.

2. The production method of enzyme of claim 1, wherein the Antrodia Cinnamomea and the fruits in the Step (a) are at a weight ratio of 1:20˜28.

3. The production method of enzyme of claim 1, wherein a Cordyceps militaris is further added in the Step (a).

4. The production method of enzyme of claim 3, wherein the Antrodia Cinnamomea and the Cordyceps militaris of the step (a) are at a weight ratio of 1:1˜2.

5. The production method of enzyme of claim 1, wherein a Curcuma longa is further added in the Step (a).

6. The production method of enzyme of claim 5, wherein the Antrodia Cinnamomea and the Curcuma longa of the step (a) are at a weight ratio of 1:0.6˜0.8.

7. The production method of enzyme of claim 1, wherein a Cordyceps militaris and a Curcuma longa are further added in the Step (a).

8. The production method of enzyme of claim 7, wherein the Antrodia Cinnamomea, the Cordyceps militaris and the Curcuma longa are at a weight ratio of 1:1˜2:0.6˜0.8.

9. The production method of enzyme of claim 1, wherein, when the detected pH value of the fermented liquid in the same container in the step (b) goes ≧3.5, the acetic acid fermentation is continued until the value reaches the pH value between 3.0˜3.5 for the solid-liquid separation.
